(19) United States
(12) Patent Application Publication
(10) Pub. No.: US 2007/0148666 A1
(43) Pub. Date: Jun. 28, 2007

(54) Diagnosis and Treatment of Brain Cancer

(76) Inventors: Rajeev Vibhakar, Iowa City, IA (US); Anup Madam, Bellevue, WA (US); Beverly L. Davidson, North Liberty, IA (US)

Correspondence Address: VIKSNINS HARRIS & PADYS PLLP, P.O. BOX 111098, ST. PAUL, MN, 55111-1098, US

(21) Appl. No.: 11/532,496

(22) Filed: Sep. 15, 2006

Related U.S. Application Data
(60) Provisional application No. 60/717,661, filed on Sep. 15, 2005.

(51) Int. Cl. C12Q 1/68; G01N 33/574

(52) U.S. Cl. 435/6; 435/7.23

(57) ABSTRACT

Certain genes are silenced in medulloblastoma. These genes include WIF1, sFRP1 and Dkk1. By detecting the expression of at least one of these genes, it is possible to diagnose cancer or to determine whether the cancer is recurring after treatment. Diagnostic methods, methods of treatment, and kits are provided.

This application claims priority to U.S. Provisional Application Ser. No. 60/717,661, filed Sep. 15, 2005, which is incorporated herein by reference in its entirety.

### BACKGROUND

DNA does not exist as naked molecules in the cell. It is associated with proteins called histones to form a complex substance known as chromatin. Chemical modifications to the DNA or the histones alter the structure of the chromatin without changing the nucleotide sequence of the DNA. Such modifications are described as “epigenetic.” Changes to the structure of the chromatin can have a profound influence on gene expression. If the chromatin is condensed, the factors involved in gene expression cannot get to the DNA, and the genes will be switched off. Conversely, if the chromatin is “open,” the genes can be switched on.

While many heritable disorders in humans are caused by DNA sequence changes (i.e., mutations) that abolish gene expression, a number of human diseases are caused by inappropriate gene silencing that is caused by epigenetic modifications. Indeed, many cancers involve the epigenetic silencing of genes that normally control cell proliferation. The major forms of epigenetic modification occurring in human tumors are DNA methylation and histone deacetylation. DNA methylation is a chemical modification of the DNA molecule itself and is carried out by an enzyme called DNA methyltransferase. Methylation can directly switch off gene expression by preventing transcription factors binding to promoters. However, a more general effect is the attraction of methyl-binding domain (MBD) proteins. These are associated with further enzymes called histone deacetylases (HDACs), which function to chemically modify histones and change chromatin structure. Chromatin-containing acetylated histones are open and accessible to transcription factors, and the genes are potentially active. Histone deacetylation causes the condensation of chromatin, making it inaccessible to transcription factors and causing the silencing of genes.

Medulloblastoma is the most common primary central nervous system tumor in childhood. Medulloblastomas arise in the cerebellum. Common symptoms are unsteadiness, headaches, and vomiting due to hydrocephalus from blockage of cerebrospinal fluid flow. Diagnosis usually occurs within one to three months of the onset of symptoms, as this is a fast-growing tumor. Occasionally, due to bleeding within the tumor, a patient will be in a coma or severe distress at diagnosis. While there has been significant improvement in survival for children with this disease, much progress needs to be made.

Histologically similar tumors may also be seen in the pineal region or the cerebral cortex. It is uncertain whether tumors which arise in these different areas of the brain are identical to medulloblastoma in their biologic behavior, as the actual cell of origin of the medulloblastoma remains unknown. Despite many investigations, the “medulloblast” has never been identified. Factors which account for the development of the medulloblastoma, its tendency to spread outside of its primary site of origin, or its ability to withstand chemotherapy and radiation are unknown.

Treatment has evolved with the majority of patients requiring surgery, radiation, and chemotherapy. Surgery for children with medulloblastoma has become safer, yet two of every ten children will develop severe, sometimes irreversible, neurologic problems afterwards, including loss of speech and severe balance difficulties. These problems may not be apparent for 24 hours after surgery and such complications have been called the “pseudobulbar syndrome” or “postcerebellar surgery mutism syndrome.” There is strong evidence that medulloblastomas that are only biopsied at the time of surgery are very difficult to control, despite the use of radiation and chemotherapy. Children whose tumors are resected (totally when possible) have a better overall rate of survival.

Radiation to the entire brain and spine at the time of diagnosis, with additional radiation (boost) to the tumor site, has been a major treatment advance. With it, the overall five-year survival rate has risen from 20% to well over 50% in the last 20 years.

Aggressive treatment approaches, especially craniospinal irradiation, can harm the developing brain. It is hard to predict what dose of radiotherapy will be harmful in each individual child. It is well known that very young children will have significant learning problems following full-dose radiotherapy, and older children may have difficulties in school. A decrease in dosage may also decrease its efficacy on the tumor. Approaches using reduced-dose craniospinal irradiation and chemotherapy, to decrease cognitive, endocrinologic, and psychological deficits, are being evaluated. They may decrease late effects, but carry with them the risk of having more disease failures.

Although currently available treatment methods alleviate symptoms temporarily, there are many cases of relapse and death within several years, with an average survival period being 15 months. The cause of relapse is believed to be that a recurrent cancer has resistance to chemotherapy and radiation.

Accurate evaluation of brain tumors relies heavily on histological techniques, and requires an extremely high level of specialized knowledge as well as auxiliary diagnostic technology. Thus, there is a pressing need for the development of diagnostic and prognostic tools and therapeutic drugs that enable early diagnosis and treatment.

### SUMMARY OF CERTAIN EMBODIMENTS OF THE INVENTION

Described herein is the finding that certain genes are silenced in medulloblastoma. These genes include WIF1, sFRP1 and Dkk1. By detecting the level of a marker expressed from at least one of these genes in a physiological sample from a subject and comparing the level to a control level, it is possible to diagnose cancer or to determine whether the cancer is recurring after treatment as a lower level of the marker in the physiological sample, as compared to the control, is indicative of the presence of the cancer.

Accordingly, certain embodiments of the present invention provide a diagnostic method for determining the presence of a brain tumor or cancer comprising comparing a test level value of at least one marker contained in a physiological sample from a subject suspected of having a brain tumor or cancer with a control level value of the at least one marker, wherein a test level value of less than the control level value is predictive of the presence of a brain tumor or cancer in the subject, and wherein the at least one marker is expressed from a nucleic acid encoding sFRP1, WIF1 or Dkk1. In certain embodiments, two markers are evaluated in the physiological sample (such as sFRP1 and WIF1, sFRP1 and Dkk1, or WIF1 and Dkk1), and in other embodiments three markers are evaluated in the physiological sample (such as sFRP1, WIF1, and Dkk1).

Certain embodiments of the present invention provide diagnostic method for predicting the recurrence of a brain tumor or cancer in a subject comprising comparing a first level value of at least one marker contained in a first physiological sample with a second level value the at least one marker from a second physiological sample, wherein a second level value of less than the first level value is predictive of the recurrence of a brain tumor or cancer in the subject, and wherein the at least one marker is expressed from a nucleic acid encoding sFRP1, WIF1 or Dkk1. In certain embodiments, two markers are evaluated in the physiological sample (such as sFRP1 and WIF1, sFRP1 and Dkk1, or WIF1 and Dkk1), and in other embodiments three markers are evaluated in the physiological sample (such as sFRP1, WIF1, and Dkk1). In certain embodiments, the first physiological sample and the second physiological sample are taken from the same patient but at different time points. The physiological samples may be taken, for example, about a week apart, about two weeks apart, about three weeks apart, about a month apart, about two months apart, about three months apart, about six months apart, or even about one year apart.

Certain embodiments of the present invention provide a diagnostic method for predicting the recurrence of a brain tumor or cancer in a subject comprising comparing a test level value of at least one marker contained in a physiological sample with a control level value of the at least one marker, wherein a test level value of less than the control level value is predictive of the recurrence of a brain tumor or cancer in the subject and wherein the at least one marker is expressed from a nucleic acid encoding sFRP1, WIF1 or Dkk1.

In some embodiments of the present invention, the tumor is a solid tumor. In some embodiments of the present invention, the tumor is a childhood tumor. In some embodiments of the present invention, the tumor is medulloblastoma.

In some embodiments of the present invention, the physiological sample is a tissue sample. In some embodiments of the present invention, the physiological sample is a fluid. In some embodiments of the present invention, the fluid is blood or cerebrospinal fluid.

In some embodiments of the present invention, the subject is a mammal. In some embodiments of the present invention, the subject is a human. In some embodiments of the present invention, the human is less than about 18 years old.

In some embodiments of the present invention, the at least one marker is a protein. In some embodiments of the present invention, the at least one marker is an RNA molecule.

In some embodiments of the present invention, the at least one marker is expressed from a nucleic acid encoding sFRP1. In some embodiments of the present invention, the at least one marker is expressed from a nucleic acid encoding WIF1. In some embodiments of the present invention, the at least one marker is expressed from a nucleic acid encoding Dkk1.

In some embodiments of the present invention, a first marker is expressed from a nucleic acid encoding sFRP1, and a second marker is expressed from a nucleic acid encoding WIF1. In some embodiments of the present invention, a first marker is expressed from a nucleic acid encoding sFRP1, and a second marker is expressed from a nucleic acid encoding Dkk1. In some embodiments of the present invention, a first marker is expressed from a nucleic acid encoding Dkk1, and a second marker is expressed from a nucleic acid encoding WIF1. In some embodiments of the present invention, a first marker is expressed from a nucleic acid encoding Dkk1, a second marker is expressed from a nucleic acid encoding WIF1, and a third marker is expressed from a nucleic acid encoding sFRP1.

Certain embodiments of the present invention provide a kit for determining the presence of a brain tumor or cancer in a subject containing packaging material and a means for detecting at least one marker that is expressed from a nucleic acid encoding sFRP1, WIF1 or Dkk1 and instructions for use as a kit for diagnosing a brain tumor or cancer in the subject.

Certain embodiments of the present invention provide a kit for predicting the recurrence of a brain tumor or cancer in a subject containing packaging material and a means for detecting at least one marker that is expressed from a nucleic acid encoding sFRP1, WIF1 or Dkk1 and instructions for use as a kit for predicting the recurrence of a brain tumor or cancer in a subject.

Certain embodiments of the present invention provide a diagnostic method for determining the presence of a brain tumor or cancer comprising comparing a test level value of at least one marker contained in a physiological sample from a subject suspected of having a brain tumor or cancer, with a control level value of the at least one marker, wherein a test level value of less than the control level value is predictive of the presence of a brain tumor or cancer in the subject, and wherein the at least one marker is expressed from a nucleic acid encoding PCDH10, ITGA7, TNFRSF19, CDKN2D (p19), CDKN2B (p15), MAP2K3, BMP4, OLIG2, PAX6, HES1, TIMP3, TPMT, sFRP1, WIF1 or Dkk1.

Certain embodiments of the present invention provide a diagnostic method for predicting the recurrence of a brain tumor or cancer in a subject comprising comparing a first level value of at least one marker contained in a physiological sample, with a second level value the at least one marker from a physiological sample, wherein a second level value of less than the first level value is predictive of the recurrence of a brain tumor or cancer in the subject, and wherein the at least one marker is expressed from a nucleic acid encoding PCDH10, ITGA7, TNFRSF19, CDKN2D (p19), CDKN2B (p15), MAP2K3, BMP4, OLIG2, PAX6, HES1, TIMP3, TPMT, sFRP1, WIF1 or Dkk1.

Certain embodiments of the present invention provide a diagnostic method for predicting the recurrence of a brain tumor or cancer in a subject comprising comparing a test level value of at least one marker contained in a physiological sample, with a control level value of the at least one marker, wherein a test level value of less than the control level value is predictive of the recurrence of a brain tumor or cancer in the subject and wherein the at least one marker is expressed from a nucleic acid encoding PCDH10, ITGA7, TNFRSF19, CDKN2D (p19), CDKN2B (p15), MAP2K3, BMP4, OLIG2, PAX6, HES1, TIMP3, TPMT, sFRP1, WIF1 or Dkk1.

Certain embodiments of the present invention provide a method for treating a subject having a brain tumor or cancer, comprising administering to the subject an effective amount of a nucleic acid encoding PCDH10, ITGA7, TNFRSF19, CDKN2D (p19), CDKN2B (p15), MAP2K3, BMP4, OLIG2, PAX6, HES1, TIMP3, TPMT, sFRP1, WIF1 or Dkk1. In some embodiments of the present invention, the nucleic acid encodes sFRP1, WIF1 or Dkk1. In some embodiments of the present invention, the nucleic acid is contained in an expression cassette and is operably linked to a promoter. In some embodiments of the present invention, the expression cassette is contained in a vector. In some embodiments of the present invention, the vector is a viral vector.

Certain embodiments of the present invention provide for the downregulation of genes that are upregulated as a consequence of the silencing of the genes disclosed herein in cancer, e.g., to treat cancer or to prevent the recurrence of cancer.

As used herein the term “a” can be mean at least one. Also, the term “or” can be used either in the disjunctive or conjunctive.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE FIGURES

FIG. 1A depicts dose response curves of cell viability for D283 medulloblastoma cells treated with increasing doses of the HDAC inhibitor TSA. FIG. 1B depicts data used to determine the optimum window at which RNA from TSA exposed cells can be harvested for microarray analysis.

FIG. 2 depicts TSA induced genes.

FIG. 3 depicts regulation of the Wnt signaling pathway (see Kawano et al., Journal of Cell Science, 116, 2627-2634 2003).

FIG. 4 depicts the TSA-indicted re-expression of Wnt antagonists.

FIG. 5 depicts data demonstrating that Wnt antagonists are expressed in mature cerebellum and normal embryonic brain but silenced in medulloblastoma cells.

FIG. 6 depicts data demonstrating that sFRP1 expression is decreased in medulloblastoma patient samples.

FIG. 7 depicts data demonstrating that sFRP1 and Dkk1 are activated by histone acetylation in medulloblastoma.

FIG. 8 depicts data demonstrating that expression of Wnt antagonists inhibits medulloblastoma tumor growth.

FIG. 9 depict data demonstrating that inhibitors of WNT signaling induce apoptosis.

FIG. 10. Demographic data in tissues, primary cell cultures and patient samples. All patient samples were obtained from Pediatric Co-operative Human Tissue Network (Columbus, Ohio). Not all samples have age and gender data available. Histology information was obtained from reports. All normal cerebellums was obtained from CHTN and originated from adult non-malignant CNS tissue.

FIGS. 11A-11C. Effects of TSA on D283 cells. (FIG. 11A) Dose response curves of cell viability for D283 medulloblastoma cells treated with increasing doses of the HDAC inhibitor TSA. (FIG. 11B) FACs analysis of TUNEL-stained (BD Biosciences) D283 cells treated with TSA for 12 hours. FACs dot plots of cells with DMSO (i), and cells treated with 0.02 μM and 0.2 μM TSA (ii and iii). (FIG. 11C) Quantification of FACs dot plots shows that 13% of DMSO treated cells are apoptotic while 31% in cells exposed to 0.02 μM TSA and 79% in 0.2 μM TSA are apoptotic.

FIGS. 12A-12B. Real time PCR validation of micro array data. FIG. 12A. Nine randomly selected genes that were statistically significant as induced at least 2-fold by TSA on micro array were analyzed by qPCR. Data is shown as Fold change of mRNA compared to DMSO (control) treated D283 cells. All genes were induced by TSA, consistent with the microarray data. FIG. 12B. Treatment with the HDAC inhibitor TSA (0.2 μM) results in induction of p21 and RASSF1 mRNA in D283 and Daoy medulloblastoma cell lines as well as MB 100 primary cells as measured by qPCR(n=3).

FIGS. 13A and 13B 13C. DKK1 expression is decreased in medulloblastoma cells and patient. FIG. 13A. Dkk1 mRNA, determined by qPCR, is significantly decreased in D283 cells and primary medulloblastoma cell cultures compared to normal human cerebellum (n=3). FIG. 13B. TSA (0.2 μm) potently induces DKK1 expression in D283 cells as determined by qPCR.

FIG. 14. Chromatin immunoprecipitation analysis of the DKK1 promoter. Histone-DNA complexes from DMSO or TSA treated D283 cells were immunoprecipitated with IgG or anti-acetylHistone (K9) antibodies and amplified with DKK-1 primers. Acetyl histone (K9) associated with DKK1 in TSA treated but not control (DMSO) treated cells. Densitometric quantification of ChIP analysis. Five fold increase in DKK1 promoter association with acetylated histone in TSA treated cells compared to DMSO controls (p<0.001).

FIG. 15. Tumor suppression by DKK1. DKK1 transfected cells produced fewer colonies than control vector on soft agar as visualized by methylene blue staining. FIG. 15A. Colony counts revel significantly less colonies in DKK1 transfected D283 cell cultures compared to control vector(n=3, p<0.001). FIG. 15C. Adenovirus mediated DKK1 expression in D283 cells leads to increased apoptosis compared to Ad-GFP controls (n=3, p<0.001)

### DETAILED DESCRIPTION

Primary brain tumors are the leading cause of death from cancer in children under the age of 15 years. Even in children cured of their brain tumors, significant treatment related morbidities arise later in life. In order to improve cure rates and decrease morbidity, better diagnostic and prognostic markers and more targeted therapies are required. Developing tumor markers and therapies that accomplish this goal requires a better understanding of the molecular mechanisms that regulate pediatric brain tumor cell growth.

Epigenetic silencing of tumor suppressor genes may play a key role in regulating the malignant potential of medulloblastoma cells by modulating proliferation, differentiation and apoptosis. Epigenetic regulation relates to heritable changes in gene expression without alterations in DNA nucleotide sequence. Histone modifications (acetylation, methylation and phosphorylation) are one of the fundamental epigenetic changes associated with transcriptional repression of genes in cancer. Deacetylation of histone proteins by histone deacetylases (HDAC) leads to compacted chromatin and transcriptional silencing . . . . Pharmacological inhibition of HDAC enzymes (e.g., by TSA) leads to histone acetylation and transcriptional activation of repressed promoters and can lead to the reactivation of tumor suppressor genes.

As described herein, medulloblastoma tumor cells were treated with TSA to uncover genes that are silenced by histone deacetylation and play a role in tumor cell growth and metastasis. Three genes in the Wnt signalling pathway were identified. These genes have not been implicated in brain cancers. Experiments in cell culture demonstrated that re-expression of the silenced genes decreases cell growth and increases cell death.

The experiments described herein indicate that genes belonging to multiple cellular processes were re-expressed by TSA-induced inhibition of HDAC in medulloblastoma cells. Further, Wnt signaling inhibitors were silenced in medulloblastoma by histone deacetylation, indicating that they may be involved in tumor pathogenesis. Also demonstrated was that the HDAC inhibitor TSA induced expression of WIF1, sFRP1 and Dkk1 in medulloblastoma cells. The inventors also demonstrated that Wnt antagonists were expressed in normal cerebellum but not medulloblastoma cells. ChIP analysis showed acetylated histones associate with sFRP1 and Dkk1 promoters. Adenoviral mediated expression of Wnt inhibitors in D283 significantly decreased colony formation on soft agar. Also, Wnt antagonists induced apoptosis in medulloblastoma cells.

Thus, the present inventors have identified and characterized novel tumor suppressor genes silenced by epigenetic mechanisms in medulloblastoma. The inventors identified molecules in the Wnt pathway that are silenced in medulloblastomas. These proteins have not yet been implicated in the control of medulloblastma growth. They were identified by microarray analyses of medulloblastoma cell lines. These genes can be used as diagnostic and/or prognostic markers.

Kits

Certain embodiments of the present invention provide kits for determining the presence of a brain tumor or cancer and kits for predicting the recurrence of a brain tumor or cancer. These kits contain packaging material and a means for detecting at least one marker that is expressed from a nucleic acid encoding sFRP1, WIF1 or Dkk1 and instructions for its intended use. In certain embodiments, the marker may be RNA and/or protein. Thus, the art worker will be able to employ methods known in the art to detect the marker. These methods include, but are not limited to, PCR and Western blotting.

Diagnostic Methods

Certain embodiments of the present invention provide methods for determining the presence of a brain tumor or cancer and methods for predicting the recurrence of a brain tumor or cancer. In certain embodiments, these methods involve the comparison of levels (“test level value”) of markers from different physiological samples. In certain embodiments, these samples can be obtained from different subjects and/or a subject and a control. In certain embodiments these samples can be obtained from the same subject at different time points, e.g., at different time points after receiving treatment for brain cancer. The markers (e.g., RNA and/or protein, and/or metabolites thereof) are expressed from genes that are downregulated in cancer. Thus, a lower level of the marker in a sample is indicative of the presence of medulloblastoma. The art worker can use assays available in the art to measure the level(s) of the marker(s).

Using the molecular markers described in this study and calculating the test level value of the marker helps predict the clinical course and eventual outcome of patients with brain tumors. Previously, the main predictors of outcome for these patients were the pathologic type and clinical stage of the tumor. Although these observations were extremely important, they did not consider the biology of the tumor. Thus the behavior of a specific tumor type may be disparate with its pathologic diagnosis. The ability to predict recurrence of disease can guide the treatment, follow-up and counseling of patients with brain tumors.

The test level value of a marker can be calculated for any brain tumor. The discovery of the predictive value of this molecular test level value of a marker is novel and overcomes the previous problem of not being able to determine the prognosis of many tumors and cancers. The test level value of a marker will vary depending on the marker tested, but an appropriate comparison to either a control test level value, or to a test level value taken from the same patient at a different time point will be easily ascertainable by one having skill in the art.

In particular, the method of the present invention may be practiced as follows. A biopsy of the tumor, or cancerous tissue or cells, is obtained. The biopsy may be from a solid tumor, such as a medulloblastoma, or a cerebrospinal fluid. The amount of a marker (e.g., protein or nucleic acid) is then quantified either directly or indirectly. For example, one way to quantify these factors is to measure the factor by quantifying the amount of RNA present using an RNase Protection Assay (RPA). Briefly, RNA is isolated from the cells in the sample, and an anti-sense RNA probe is hybridized in excess to the target RNA in solution. Free probe and non-hybridized single stranded RNA were digested with RNases. Labeled anti-sense RNA is transcribed incorporating a labeled nucleotide, such as an [alpha-³²P]UTP. Total RNA is extracted from sample. The amount of labeled mRNA was then quantified. Other methods to quantify the test marker of interest include by Western blot or by immunohistochemical staining. Useful ligands for detecting the marker may be an antibody, such as a monoclonal antibody or a population of polyclonal antibodies. Other RNA and protein detection methods are well-known to those with skill in the art.

Thus, the present inventors have discovered that a decrease in certain cell markers is predictive of the presence of a brain tumor or caner, or the recurrence of a brain tumor or cancer in the patient. The inventors have found that the calculation of the test level value of certain markers is a powerful predictor of the presence or recurrence of disease, and assists in guiding treatment, counseling and follow-up therapeutic strategies with patients with tumors. The test level value of the sample may be less than the control level value by about 10%, 20%, 30%, 40%, 50%, 60%, 70%, 80%, 90%, 100%, 200%, 300%, 400%, 500%, 600%, 700%, 800%, 900%, 100%, 1500% or even 2000% or more.

The present invention provides a diagnostic method for predicting the presence or recurrence of a tumor or cancer in a mammal by contacting RNA from a mammalian physiological sample suspected of being tumorigenic or cancerous with a labeled marker-specific oligonucleotide under conditions effective to hybridize the RNA to the oligonucleotide so as to yield a population of RNA labeled with the marker-specific oligonucleotide; quantifying the population of labeled RNA to determine an amount of marker RNA present in the sample; and comparing the amount of maker to a control level of marker; wherein a lesser amount of marker in the sample as compared to the control level is predictive of the presence of a tumor, or that the tumor will recur. The marker may be PCDH10, ITGA7, TNFRSF19, CDKN2D (p19), CDKN2B (p15), MAP2K3, BMP4, OLIG2, PAX6, HES1, TIMP3, TPMT, sFRP1, WIF1 or Dkk1. The physiological sample may be a tissue sample, such as a biopsy, or a spinal fluid sample. The tissue may be any solid tumor, encompassing childhood and adult tumors. Alternatively, instead of comparing the amount of marker in the sample to a control level of marker, one can take two samples from the patient at different time points, and determine the test level of the marker at the two times, where a decrease in the level of marker over time is predictive of the tumor recurring.

The present invention also provides a diagnostic method for predicting the presence recurrence of a tumor or cancer in a mammal by contacting a mammalian tissue sample suspected of being tumorigenic or cancerous with a labeled marker-specific ligand under conditions effective to hybridize marker-protein present in the tissue sample to the ligand so as to yield a population of protein hybridized to the marker-specific ligand; and comparing the amount of maker to a control level of marker; wherein a lesser amount of marker in the sample as compared to the control level is predictive of the presence of a tumor, or that the tumor will recur. The physiological sample may be a tissue sample, such as a tissue-lysate protein sample. The physiological sample may be a tissue sample, such as a biopsy, or a spinal fluid sample. The tissue may be any solid tumor, encompassing childhood and adult tumors. The ligand may be an antibody, in particular a population of polyclonal or monoclonal antibodies. Alternatively, instead of comparing the amount of marker in the sample to a control level of marker, one can take two samples from the patient at different time points, and determine the test level of the marker at the two times, where a decrease in the level of marker over time is predictive of the tumor recurring.

Detection Labels

The labels used in the assays of invention can be primary labels (where the label comprises an element that is detected directly) or secondary labels (where the detected label binds to a primary label, e.g., as is common in immunological labeling). An introduction to labels (also called “tags”), tagging or labeling procedures, and detection of labels is found in Polak and Van Noorden (1997) Introduction to Immunocytochemistry, second edition, Springer Verlag, N.Y. and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetramethylrhodamine isothiocyanate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P), enzymes (e.g., horse-radish peroxidase, alkaline phosphatase) spectral colorimetric labels such as colloidal gold or colored glass or plastic (e.g. polystyrene, polypropylene, latex) beads. The label may be coupled directly or indirectly to a component of the detection assay (e.g., the labeled nucleic acid) according to methods well known in the art. As indicated above, a wide variety of labels may be used, with the choice of label depending on sensitivity required, ease of conjugation with the compound, stability requirements, available instrumentation, and disposal provisions. In general, a detector that monitors a probe-substrate nucleic acid hybridization is adapted to the particular label that is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeled nucleic acids is digitized for subsequent computer analysis.

Preferred labels include those that use (1) chemiluminescence (using Horseradish Peroxidase and/or Alkaline Phosphatase with substrates that produce photons as breakdown products) with kits being available, e.g., from Molecular Probes, Amersham, Boehringer-Mannheim, and Life Technologies/Gibco BRL; (2) color production (using both Horseradish Peroxidase and/or Alkaline Phosphatase with substrates that produce a colored precipitate) (kits available from Life Technologies/Gibco BRL, and Boehringer-Mannheim); (3) hemifluorescence using, e.g., Alkaline Phosphatase and the substrate AttoPhos (Amersham) or other substrates that produce fluorescent products, (4) Fluorescence (e.g., using Cy-5 (Amersham), fluorescein, and other fluorescent labels); (5) radioactivity using kinase enzymes or other end-labeling approaches, nick translation, random priming, or PCR to incorporate radioactive molecules into the labeled nucleic acid. Other methods for labeling and detection will be readily apparent to one skilled in the art.

Fluorescent labels are highly preferred labels, having the advantage of requiring fewer precautions in handling, and being amendable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Fluorescent moieties, which are incorporated into the labels of the invention, are generally are known, including Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes, flavin and many others. Many fluorescent labels are commercially available from the SIGMA Chemical Company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka ChemicaBiochemika Analytika (Fluka Chemie AG, Buchs, Switzerland), and Applied Biosystems (Foster City, Calif.), as well as many other commercial sources known to one of skill.

Means of detecting and quantifying labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems that are widely available.

Treatments

Certain embodiments of the present invention provide methods for treating a subject having a brain tumor or cancer, including administering to the subject an effective amount of a nucleic acid encoding a protein that is downregulated in medulloblastoma. In some embodiments of the invention, the gene that is downregulating is at least one of sFRP1, WIF1 or Dkk1.

The general methods for constructing vectors that can transform host cells are well known to those skilled in the art, and the same compositions and methods of construction may be utilized to produce the somatic cell gene targeting vectors described herein. For example, suitable methods of construction are disclosed in Sambrook and Russell, Molecular Cloning: A Laboratory Manual, 3rd ed., Cold Spring Harbor Laboratory Press, Cold Spring Harbor, N.Y. (2001).

The term “transformation” refers to the transfer of a nucleic acid fragment into the genome of a host cell, resulting in genetically stable inheritance. “Transformed,” “transgenic,” or “recombinant” refer to a host cell into which a heterologous nucleic acid molecule has been introduced through the transformation process. Nucleic acid molecules can be stably integrated into a host cell's genome using techniques generally known in the art (Sambrook and Russell, 2001). The term “untransformed” refers to normal cells that have not been through the transformation process.

“Chromosomally-integrated” refers to the integration of a foreign gene or DNA construct into the genomic DNA (i.e., genome) of the host cell by covalent bonds. “Genome” refers to the complete genetic material of an organism.

A “host cell” is a cell that has been transformed or a cell that is capable of transformation by an exogenous nucleic acid molecule. In particular, host cells of the present invention are somatic cells, e.g., a B cell or a macrophage. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

A gene targeting vector of the invention can have a gene targeting construct, which can include, inter alia, cloning sites, DNA encoding a selectable marker and/or a polyadenylation sequence that are operably linked using techniques known to the art (Sambrook and Russell, 2001), as well as an expression cassette with a negatively selectable marker.

“Operably linked” nucleic acids are nucleic acids placed in a functional relationship with another nucleic acid sequence, e.g., DNA sequences linked on single nucleic acid fragment so that the function of one is affected by the other. For example, in the expression cassette of the invention, the functional linkage of a regulatory sequence, e.g., a promoter, is functionally linked to a heterologous nucleic acid sequence, e.g., DNA encoding a negatively selectable marker, resulting in expression of the latter. As another example, a promoter is operably linked to a coding sequence if it affects the transcription of the sequence. Generally, operably linked DNA sequences are DNA sequences that are contiguous. Linking is accomplished by ligation at convenient restriction sites. If such sites do not exist, synthetic oligonucleotide adaptors or linkers are used in accord with conventional practice. Additionally, multiple copies of the nucleic acid may be linked together in the expression vector. Such multiple nucleic acids may be separated by linkers.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

Generally, a vector of the present invention is a replicon, such as a plasmid, phage, virus, or cosmid, to which another DNA segment may be attached so as to bring about the replication of the attached segment. A “vector” is therefore, defined to include, inter alia, any plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Expression cassette” as used herein means a DNA sequence capable of directing expression of a particular nucleotide sequence in an appropriate host cell, comprising a promoter operably linked to the nucleotide sequence of interest that is operably linked to termination signals. It also typically includes sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “introns” refers to a DNA sequence present in a given gene which is not translated into protein and is generally found between exons.

As used herein, “DNA” encompasses nucleic acids that are deoxyribonucleotides and polymers thereof in either single- or double-stranded form, composed of monomers (nucleotides) containing a sugar, phosphate and a base that is either a purine or pyrimidine. Unless specifically limited, the term DNA encompasses nucleotides containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

The use of “promoterless” constructs for the gene targeting constructs of the invention is desired. By “promoterless” is meant that the construct does not contain a promoter. As discussed herein, use of this feature makes expression of the constructs of the invention conditional upon homologous recombination at the targeted locus. The vector in which the construct is contained may have a promoter, but the promoter is not contained in the construct portion of the vector. Promoterless vectors are known in the art (see, for example, Sedivy and Dutriaux, 1999).

“Expression” refers to the transcription and/or translation of an endogenous gene or a transgene in cells.

A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “protein,” “peptide” and “polypeptide” are used interchangeably herein).

In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention.

By “fragment,” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment,” means, for example, a sequence having at least 80 nucleotides, at least 150 nucleotides, or at least 400 nucleotides. If not employed for expressing, a “portion” or “fragment” means, for example, at least 9, at least 12, at least 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

In general, the invention relates to polynucleotides, polypeptides, and vectors, and the use of them. In particular, the invention relates to a method for gene or protein therapy that is capable of both localized and systemic delivery of a therapeutically effective dose of a therapeutic agent.

Expression vectors of the instant invention include, but are not limited to, viruses, plasmids, and other vehicles for delivering heterologous genetic material to cells. Accordingly, the term “expression vector” as used herein refers to a vehicle for delivering heterologous genetic material to a cell. In particular, the expression vector is a recombinant adenoviral, adeno-associated virus, or lentivirus or retrovirus vector.

The expression vector further includes a promoter for controlling transcription of the heterologous gene. The promoter may be an inducible promoter (described below). The expression system is suitable for administration to the mammalian recipient.

According to yet another aspect of the invention, a method for treating a mammalian recipient in vivo is provided. The method includes introducing an expression vector for expressing a heterologous gene product into a cell of the patient in situ. To form the expression system in vivo, an expression vector for expressing the therapeutic agent is introduced in vivo into target location of the mammalian recipient by, for example, intraperitoneal injection or injection directly into the brain.

The expression vector for expressing the heterologous gene may include an inducible promoter for controlling transcription of the heterologous gene product. Accordingly, delivery of the therapeutic agent in situ is controlled by exposing the cell in situ to conditions, which induce transcription of the heterologous gene.

The present invention provides methods of treating a tumor or cancer in a subject by administering a polynucleotide, polypeptide, or expression vector. For the gene therapy methods, a person having ordinary skill in the art of molecular biology and gene therapy would be able to determine, without undue experimentation, the appropriate dosages and routes of administration of the polynucleotide, polypeptide, or expression vector used in the novel methods of the present invention.

The term “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence which works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

The selection and optimization of a particular expression vector for expressing a specific gene product in an isolated cell is accomplished by obtaining the gene, potentially with one or more appropriate control regions (e.g., promoter, insertion sequence); preparing a vector construct comprising the vector into which is inserted the gene; transfecting or transducing cultured cells in vitro with the vector construct; and determining whether the gene product is present in the cultured cells.

In one embodiment, vectors for cell gene therapy are viruses, such as replication-deficient viruses. Exemplary viral vectors are derived from: Harvey Sarcoma virus; ROUS Sarcoma virus, (MPSV); Moloney murine leukemia virus and DNA viruses (e.g., adenovirus) (Temin, H., “Retrovirus vectors for gene transfer”, in Gene Transfer, Kucherlapati R, Ed., pp 149-187, Plenum, (1986)).

The major advantage of using retroviruses, including lentiviruses, for gene therapy is that the viruses insert the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. While proliferation of the target cell is readily achieved in vitro, proliferation of many potential target cells in vivo is very low.

Yet another viral candidate useful as an expression vector for transformation of cells is the adenovirus, a double-stranded DNA virus. Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Finally, a third virus family adaptable for an expression vector for gene therapy are the recombinant adeno-associated viruses, specifically those based on AAV2, AAV4 and AAV5 (Davidson et al, PNAS, 2000).

Thus, as will be apparent to one of ordinary skill in the art, a variety of suitable viral expression vectors are available for transferring exogenous genetic material into cells. The selection of an appropriate expression vector to express a therapeutic agent for a particular condition amenable to gene replacement therapy and the optimization of the conditions for insertion of the selected expression vector into the cell, are within the scope of one of ordinary skill in the art without the need for undue experimentation.

In an alternative embodiment, the expression vector is in the form of a plasmid, which is transferred into the target cells by one of a variety of methods: physical (e.g., microinjection), electroporation, scrape loading, microparticle bombardment or by cellular uptake as a chemical complex (e.g., calcium or strontium co-precipitation, complexation with lipid, complexation with ligand) (Methods in Molecular Biology, Vol. 7, Gene Transfer and Expression Protocols, Ed. E. J. Murray, Humana Press (1991)). Several commercial products are available for cationic liposome complexation including Lipofectin™ (Gibco-BRL, Gaithersburg, Md.) (Felgner, P. L., et al., Proc. Natl. Acad. Sci. 84:7413-7417 (1987)) and Transfectam™ (ProMega, Madison, Wis.). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation.

Therefore, the following examples are intended to illustrate but not limit the invention.

### EXAMPLE 1

### HDAC Inhibition Decreases Medulloblastoma Cell Viability

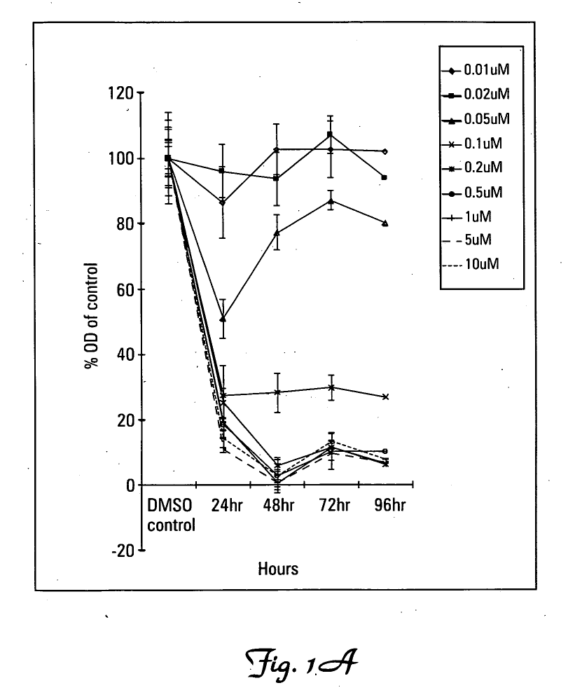

The inventors tested cell viability of D283 medulloblastoma cells treated with increasing doses of the HDAC inhibitor TSA (FIG. 1A). They also tested cell viability was measured by an MTT assay. TSA concentrations of 0.1 μM and 0.2 μm decreased viability to 50% and 30% at 24 h respectively.

The inventors also determined the minimum time of TSA exposure needed to induce cell death (FIG. 1B). D283 cells were exposed to TSA for various time points and the media replaced for an additional 24 h prior to assaying for cell viability. Treatment between 6 and 9 h resulted in 80% surviving cells. These data provided the optimum window at which RNA from TSA exposed cells can be harvested for microarray analysis.

### EXAMPLE 2

### Genome Wide Analysis of Epigenetic Gene Silencing Implicates Multiple Cellular Pathways in Medulloblastoma Pathogenesis

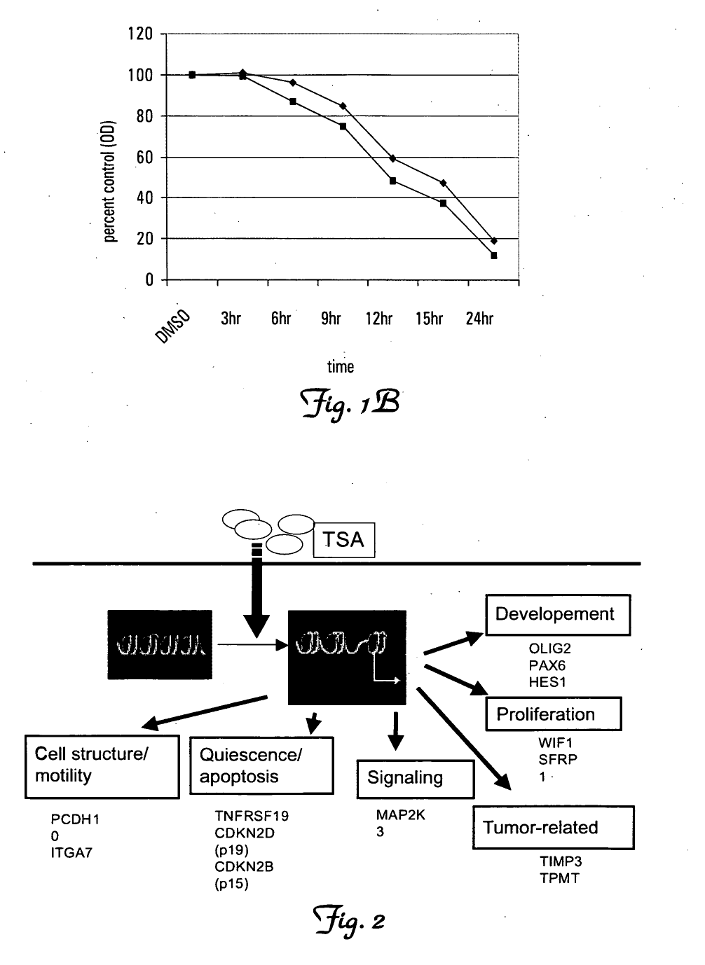

The inventors found that TSA induced 714 genes at least 2-fold representing multiple cellular pathways (FIG. 2). RNA from vehicle and TSA treated D283 cells was reverse-transcribed and hybridized to the ABI 1700 Human Genome Expression Microarray containing 33,202 gene-specific 60-mer oligonucleotide probes. Data was quantile normalized and t-test was applied to each gene for statistical significance. Differential gene expression was quantified using the Storey q-value method and data visualized using Spotfire software.

### EXAMPLE 3

### Antagonists of Wnt Signalling are Epigenetically Silenced in Medulloblastoma

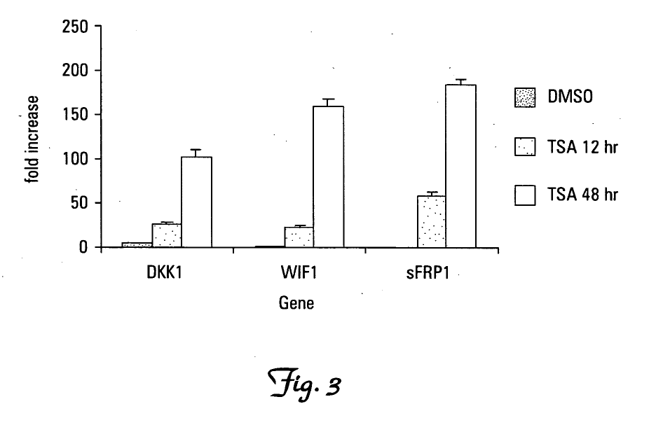

The inventors performed studies that determined that antagonists of Wnt signalling are epigenetically silenced in medulloblastoma. Table 1 shows that inhibitors of WNT signaling are up-regulated by TSA in medulloblastoma (FIG. 3).

TABLE 1

| Gene | TSA fold increase |
|---|---|
| sFRP 1 | 2.9 |
| WIF 1 | 2.5 |
| Dkk 1 | 1.4 |

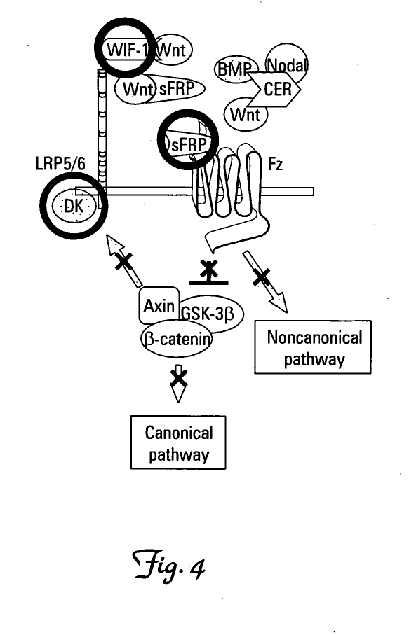

Clustering of genes reveals multiple signal transduction pathways affected by epigenetic silencing in medulloblastoma. Bioinformatic analysis reveals three of these genes to be regulators of the pro-proliferative WNT signaling pathway as shown in FIG. 4.

### EXAMPLE 4

### Real time PCR Confirms TSA-Induced Re-Expression of Wnt Antagonists

The inventors identified genes by means of the microarray analysis. The results were verified by real time PCR (ABI) in D283 cells using Assay on demand gene expression reagents (ABI) for selected genes. Human Gus was used as the normalizer gene. Real-time PCR was performed on ABI PRISM 7900 HT detection system. Validated genes were assayed in normal brain and medulloblastoma patient samples.

### EXAMPLE 5

### Wnt Antagonists are Expressed in Human Cerebellum but not Medulloblastoma Cells

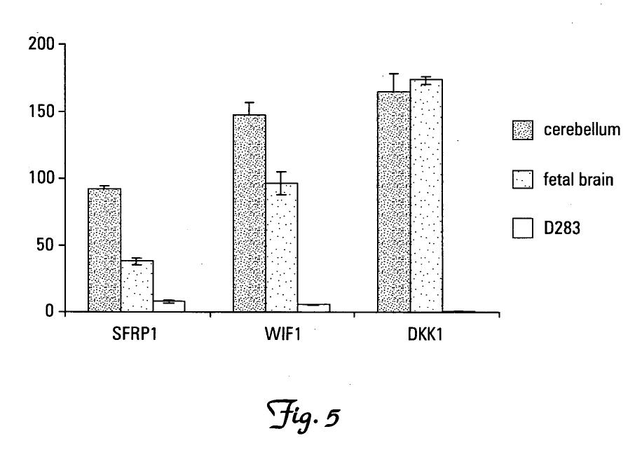

Wnt antagonists are expressed in mature cerebellum and normal embryonic brain but silenced in D283 medulloblastoma cells (FIG. 5). Expression of wnt inhibitors sFRP1, WIF1 and Dkk1 was measured by real time PCR on an ABI PRISM 7900 HT detection system and normalized to the human Gus gene. Each experiment was carried out in triplicate.

### EXAMPLE 6

### sFRP1 is Silenced in Medulloblastoma Patient Samples

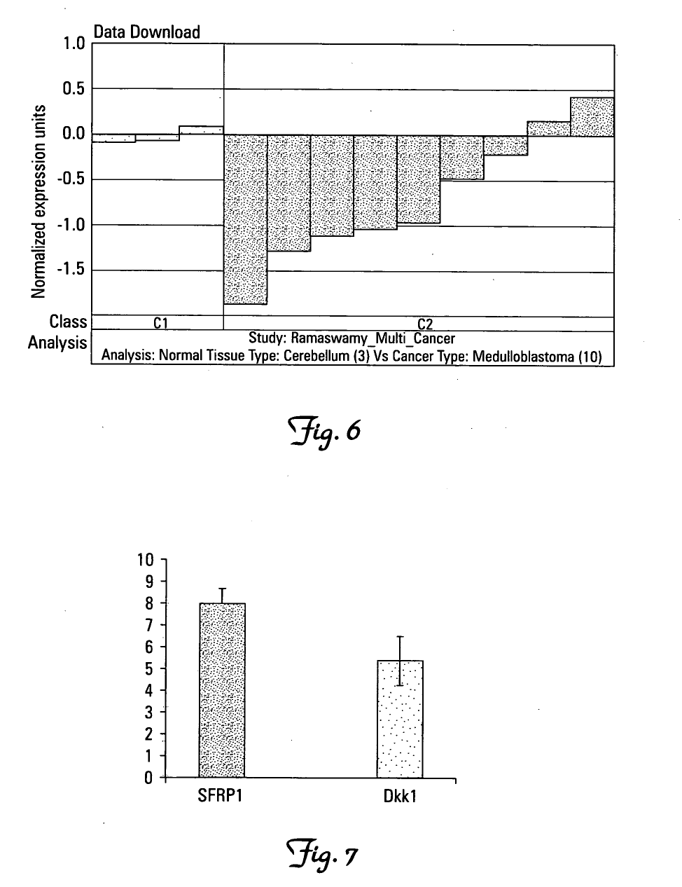

Bioinformatic analysis of public databases reveals sFRP1 expression to be decreased in medulloblastoma patient samples (FIG. 6). The Oncomine cancer microarray database (on the World-Wide-Web at oncomine.org) was mined for Wnt inhibitor expression in medulloblastoma. One microarray study included 10 medulloblastoma samples with data on sFRP1 expression. sFRP1 was significantly decreased in expression in 8/10 patient samples compared to normal cerebellum.

### EXAMPLE 7

### Chromatin Immunoprecipitation Indicates that sFRP1 and Dkk1 are Activated by Histone Acetylation in Medulloblastoma

ChIP assays confirm that sFRP1 and WIF1 are activated by histone acetylation. In these assays control (DMSO) or TSA treated D283 cells were incubated with formaldehyde to crosslink histones to DNA. Cell lysates were incubated with protein A-agarose beads followed by centrifugation. The supernatant was and incubated at 4° C. overnight in four parts (input control, anti-K9 acetylated histone H3, normal Rabbit IgG or no antibody (negative control)). The immunoprecipitated complexes were collected, washed and the cross links were reversed. The DNA was extracted for PCR. Primers were designed to amplify 250 bp promoter region. PCR products were quantified by densitometry and plotted as a ratio of acetylated histone (TSA treatment) to un-acetylated histone (DMSO treatment) (FIG. 7).

### EXAMPLE 8

### Expression of Wnt Antagonists Inhibits Medulloblastoma Tumor Growth

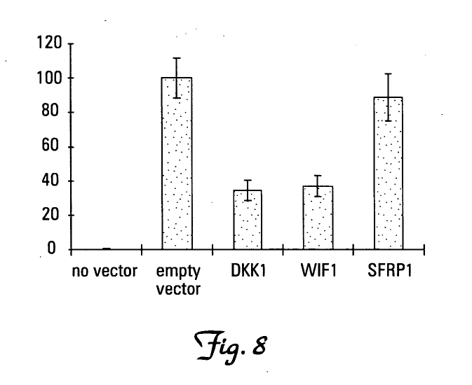

Colony focus assays demonstrated that Wnt antagonists inhibit D283 tumor growth. Wnt inhibitors were cloned into pcDNA3.1, D283 cells transfected and plated on agar. Colonies selected in G418. Total number of colonies formed on agar was counted for three separate experiments (FIG. 8).

### EXAMPLE 9

### Induction of Apoptosis by WNT Inhibitors

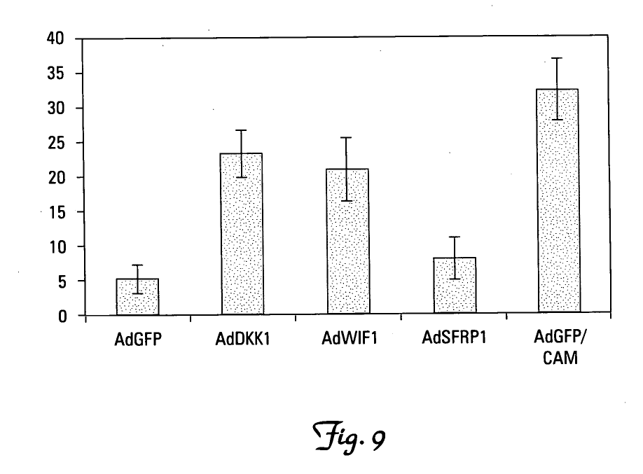

The inventors tested inhibitors of WNT signaling induce apoptosis in medulloblastoma cells. D283 cells were infected with Adenoviral vectors expressing GFP alone or a WNT inhibitor plus GFP. Camptothecin (CAM) was used as a positive control for induction of apoptosis. Apoptosis was assayed 48 hours post infection by FACS analysis of Anexin V staining (BD Pharmingen, San Diego) (FIG. 9). Mean of 4 separate experiments is displayed.

### EXAMPLE 10

### Dickkopf-1 is an Epigenetically Silenced Candidate Tumor Suppressor Gene in Medulloblastoma

Medulloblastoma accounts for approximately 20% of all primary pediatric brain tumors. Although advances in treatment with surgery, radiation and chemotherapy have increased the five-year survival rate to approximately 70%, children younger than three years of age show significantly worse outcomes. Current medulloblastoma treatments have devastating morbidity associated with them because they lack specificity thus new approaches are needed. Understanding the molecular basis of medulloblastoma pathogenesis may identify signaling pathways for targeted therapy. Recent advances have identified several genetic mechanisms such as mutations and loss of hetrozygosity leading to tumor suppressor loss in medulloblastoma. However other mechanisms of tumor suppressor loss have not been extensively studied in medulloblastoma.

Over the past several years there has been an increasing realization that many tumor suppressor genes are silenced by epigenetic rather than genetic mechanisms. Disruption of epigenetic mechanisms is considered to be closely linked to aberrant expression of cancer associated genes. Two fundamental epigenetic changes are associated with transcriptional repression of genes in cancer. These are histone modifications (acetylation, methylation and phosphorylation) and hypermethylation of CpG motifs in DNA promoter regions. Abundant evidence supports a closed interplay between DNA methylation and histone modifications for establishing gene silencing. Several recent reports indicate that changes in histone tail modifications can overcome the repressive barrier of DNA methylation. This has led to the hypothesis that changes in chromatin remodeling proteins are the primary event in creating a “closed” local chromatin structure associated with repressed transcriptional activity of genes. While there are several reports of DNA methylation in medulloblastoma, the role of histone modifications in regulating gene expression in medulloblastoma has not previously been described. An extensive characterization of genes silenced due to pathological changes in chromatin structure in medulloblastoma could offer a better chance for curative measures.

In the present study, the inventors sought to identify genes activated through pharmacological reversal of histone de-acetylation by Trichostatin A (TSA) in medulloblastoma cells using whole genome microarray analysis. TSA is a potent histone deacetylase (HDAC) inhibitor. The inventors identified DKK1 as significantly up-regulated on HDAC inhibition. The inventors confirmed transcriptional silencing of DKK1 in the D283 cell line, and more important, in patient derived primary medulloblastoma cells as well as a panel of tumor tissues. Histone acetylation in the promoter region of DKK1 increased 5-fold in response to HDAC inhibition. Re-expressing DKK1 in medulloblastoma cells induced apoptosis and inhibited clonogenic growth supporting its role in the control of cell growth. These data demonstrate the importance of histone acetylation in regulating gene expression in medulloblastoma, and implicate the dysregulation of DKK1 as a potential component of medulloblastoma pathogenesis.

Materials and Methods

Cells, Tissues and Culture

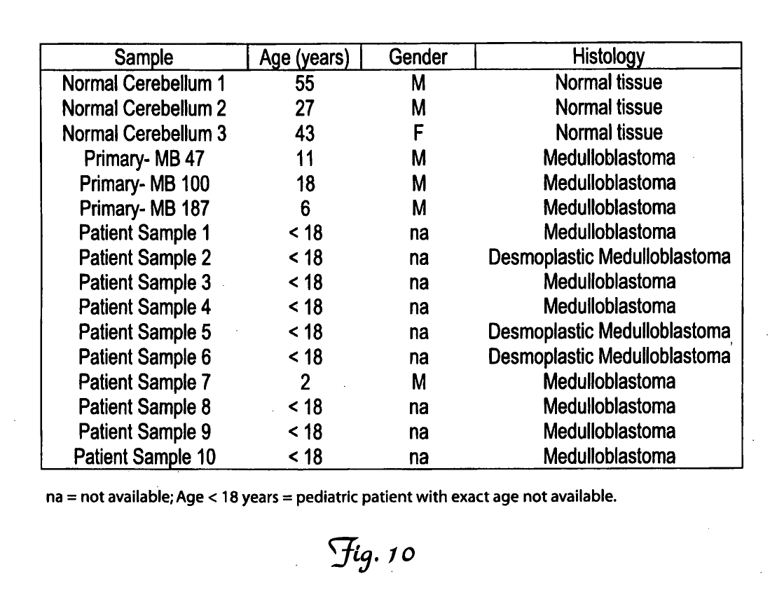

D283 medulloblastoma cells were obtained from American Type Culture Collection (Rockville, Md.) and cultured in modified Eagles Medium (Gibco, Grand Island, N.Y.) supplemented with 10% fetal bovine serum (Gibco, Grand Island, N.Y.) according to the supplier's recommendations. Primary cell cultures were derived from biopsy specimens of medulloblastoma patients under an IRB approved protocol at the University of Iowa Hospitals and Clinics. To generate primary cell cultures, approximately 200 to 250 mg of tumor tissue was immersed and incubated in 0.05 mM EDTA solution containing 0.05% trypsin (Sigma, St. Louis, Mo.) at 4° C. for 8 hours. The tissue samples were minced into 0.3 mm³ fragments and suspended in HBSS containing 4 mg of DNaseI, 40 mg of collagenase IV, and 100 units of hyaluronidase type V (all from Sigma, St. Louis, Mo.). Single-cell suspensions were then passed through No. 100 nylon mesh, washed twice in HBSS, and added to fibronectin-coated tissue culture flasks. Cultures were maintained at low passage numbers (p2-4) in modified Eagles Medium supplemented with 10% fetal bovine serum as described above. Normal human cerebellum and medulloblastoma patient samples were obtained from the Pediatric Co-operative Human Tissue Network (Columbus, Ohio). All normal cerebellar samples were from non malignant adult brain. All medulloblastoma samples were from pediatric patients (<18 years of age). Detailed data on the normal samples, primary cultures and patient samples can be found in FIG. 10.

Microarray Analysis

The D283 cell line was cultured with either 0.2 μM TSA or DMSO for 9 hours to generate gene expression profiles in response to TSA. Total RNA was extracted from treated cells using Trizol (Invitrogen, Carlsbad, Calif.). RNA was further purified using the RNeasy kit (QIAGEN, Valencia, Calif.) per manufacturer's protocol and purity of RNA was determined by the Agilent Bioanalyzer. 2 μg of total RNA was reverse-transcribed with the Chemiluminescent RT-IVT Labeling Kit (Applied Biosystems, Foster City, Calif.) and hybridized to a 60-mer whole genome oligonucleotide microarray (Applied Biosystems, Foster City, Calif.) containing 33,202 probes representing 29,098 genes per manufacturer's protocol. A total of three microarray hybridizations, one for each biological replicate, were performed per treatment. Data was quantile normalized and t-test was applied to each gene for statistical significance. Differential gene expression was quantified using Storey q-value method (Storey and Tibshirani, 2003, Proc Natl Acad Sci USA 100, 9440-9445). Spotfire software was used for data visualization and a cut-off of 2-fold threshold with a false discovery rate of 1% was used to identify epigenetically regulated genes. Assay on Demand gene expression reagents (Applied Biosystems, Foster City, Calif.) for nine randomly selected genes were used to validate microarray data. Data were submitted to the NCBI gene expression omnibus (GEO) database (on the World Wide Web at ncbi.nlm.nih.gov/geo).

Real-Time Quantitative RT-PCR

RNA was isolated from cells and tissues with Triazol (Invitrogen, Carlsbad, Calif.). Real-time PCR was performed on the ABI PRISM 7900 HT detection system using Taq man reagents (Applied Biosystems, Foster City, Calif.) per the manufacturer's recommendations. Gene expression was determined with Assay on Demand gene expression reagents (Applied Biosystems, Foster City, Calif.). All assays were done in triplicate.

Chromatin Immunoprecipitation

ChIP analysis was done using primary antibodies to acetylated histone 3 (Upstate biotechnology, Lake Placid, N.Y.). Control (DMSO) or TSA treated D283 cells (1×10⁶) were incubated with 1% formaldehyde for 10 minutes to crosslink histones to DNA. Cells were washed with cold PBS and resuspended in lysis buffer (Upstate Biotechnology, Lake Placid, N.Y.) and sonicated for 10 sec with continuous output using a Branson sonifier. The lysate was centrifuged for 10 minutes at 13,200 rpm at 4° C. after which the supernatant was incubated with protein A-agarose beads (Upstate Biotechnology, Lake Placid, N.Y.) for two hours. The slurry was removed by centrifugation at 1000 rpm for 1 minute. The supernatant was collected and incubated at 4° C. overnight in four parts (input control, anti-K9 acetylated histone H3, normal Rabbit IgG or no antibody). The immunoprecipitated complexes were collected, washed and the cross links reversed. The samples were then treated with proteinase K overnight and DNA was extracted by the phenol chloroform method, ethanol precipitated, and re-suspended in 50 μl of water. PCR was performed on extracted DNA using primers designed to amplify a 250 bp promoter region. To ensure that PCR amplification was in linear range, each reaction was set up at different dilutions of DNA for varying amplification cycle numbers and final PCR conditions were selected accordingly. The PCR mixture contained 20 pM of each primer, 1 μl of extracted DNA, 0.5 units of Taq DNA Polymerase (Eppendorf, Pittsburg, Pa.), 0.2 mM dNTPs (each) and 2 mM MgSO₄ in a final volume of 50 μl. The PCR was performed with the following cycling parameters: an activation step of 94° C. for 3 min followed by 30 cycles of 94° C. for 2 min, 50° C. for 2 min and 68° C. for 3 min, with a final extension step of 68° C. for 10 min. The promoter region of DKK1 was amplified and the PCR products were quantified by densitometry and plotted as a ratio of acetylated histone (TSA treatment) to un-acetylated histone (DMSO treatment). The assays were done in triplicate.

Construction of Expression Vectors

Full-length open reading frame for DKK1 was PCR amplified from a Mammalian Gene Collection clone (MGC:868, BC001539), subcloned into the pcDNA3.1 D/V5-His-TOPO vector (Invitrogen) and the sequence verified. The PCR product was also cloned into pAD5CMVIRESeGFPpA, its sequence verified. The clone was recombined in HEK293 cells with pacAD5 9.2-100 to produce recombinant adenovirus particles (University of Iowa Gene Transfer Vector Core).

Transfection and Colony Formation Assays

Colony formation assays were performed on soft agar. Cells were plated at 1.5×10⁵ per well using 6-well plates and transfected with pcDNA3.1D/V5-His-TOPO/DKK1, pcDNA3.1D/N5-His-TOPO/lacZ or pcDNA3.1D/V5-His-TOPO with no insert (mock control) using Trans It-Neural transfection reagents (Mirus). The cells were selected in G418 (1 mg/ml) supplemented media at 24 hr post transfection. The cells were simultaneously harvested to confirm their expression at the mRNA level by real-time PCR. G418 resistant cells were maintained for two weeks in culture. Cells were re-suspended in media containing 0.3% agarose and were overlaid on 0.6% agarose. 0.5 ml media was added to the plates every four days and colony formation was quantified after fixation and staining with methylene blue after 3 weeks.

Apoptosis Assay

Apoptosis was measured by anexin staining. Control or infected cells were incubated with Anexin-PE antibody (BD Pharmingen, San Diego, Calif.) and counterstained with 7-AAD per the manufacturer's protocol. Cell fluorescence was measured on a FACScan flowcytometer (BD, San Diego, Calif.) and analyzed with FlowCyto software.

Results

HDAC Inhibition in Medulloblastoma Cells Induces Expression of Genes Involved in Varying Biological Processes

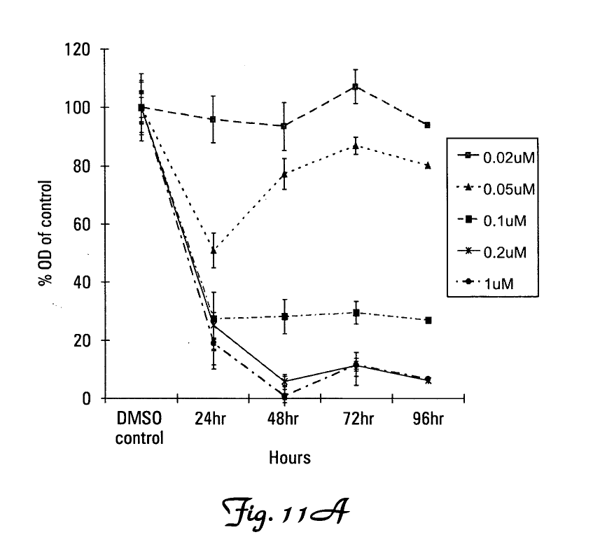

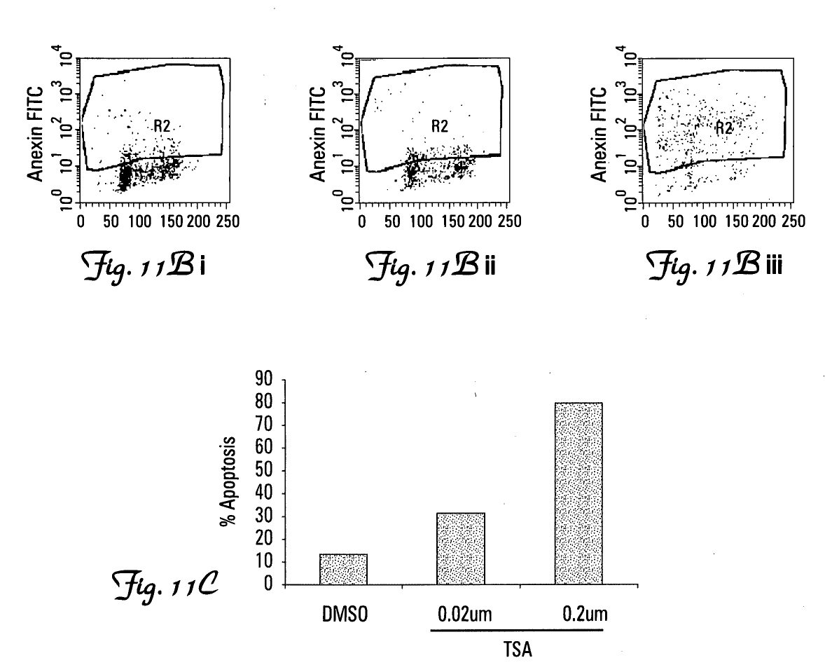

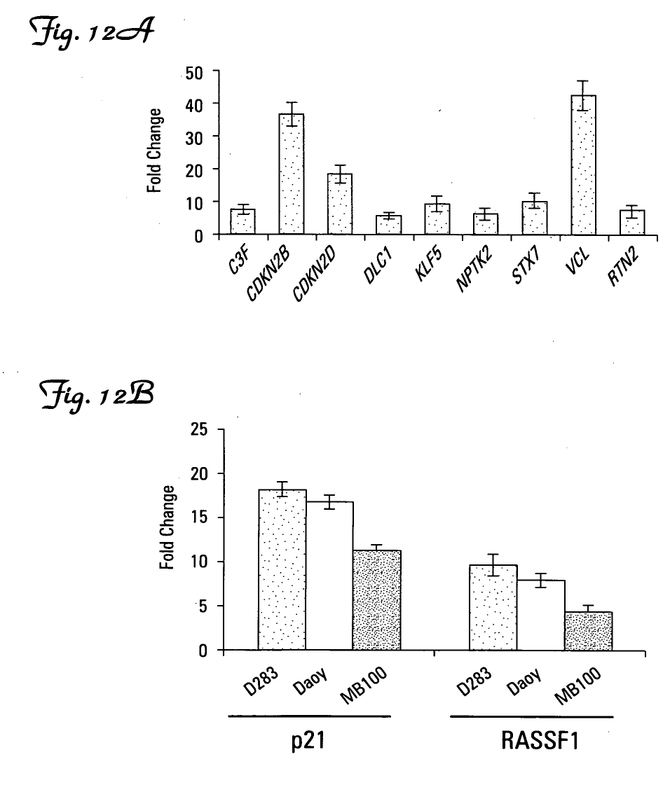

To identify genes regulated by changes in histone H3 K9 acetylation status, the inventors first determined the optimal dose and timing for treating D283 medulloblastoma cells with TSA. The D283 cell line was chosen since it is widely used as a cell model of medulloblastoma and is well characterized. TSA potently decreased D283 medulloblastoma cell viability and induced apoptosis (FIGS. 11A-11C). For microarray studies, the inventors treated D283 cells with 0.2 μM TSA for 9 hours. The dose and time point was chosen based on viability assays and washout experiments. At this concentration cell viability is 100% but the majority (85%) of cells have committed to cell death by 24 hours. In addition, 9 hours of TSA exposure results in robust histone acetylation as measured by western blot analysis. Whole genome analysis revealed 714 genes were up regulated by TSA at least two fold at a maximal statistical stringency (q<0.001). To confirm the microarray analysis, real-time quantitative PCR (qPCR) was performed on nine randomly selected genes (FIG. 12A). We then demonstrated that the effects of TSA on induction of gene expression are operative in additional medulloblastoma cell lines. TSA treatment induced expression of p21 and RASSF1 in D283 and Daoy medulloblastoma cell lines and in MB100 primary cell cultures. Both P21 and RASSF1 have been previously identified as genes induced by TSA. We next analyzed the functional significance of the up-regulated genes by mapping them to various pathways using the PANTHER classification system (on the World Wide Web at pantherdb.org). Of the 714 genes up regulated at least 2 fold, 106 mapped to 68 known signaling pathways (Table 2).

TABLE 2

| | Number of |
|---|---|
| Pathway | Genes |
| Adrenaline and noradrenaline biosynthesis | 3 |
| Alpha adrenergic receptor signaling pathway | 2 |
| Alzheimer disease-amyloid secretase pathway | 1 |
| Alzheimer disease-presenilin pathway | 2 |
| Aminobutyrate degradation | 2 |
| Angiogenesis | 14 |
| Apoptosis signaling pathway | 6 |
| Axon guidance mediated by netrin | 6 |
| Axon guidance mediated by semaphorins | 4 |
| Axon guidance mediated by Slit/Robo | 1 |
| B cell activation | 5 |
| Blood coagulation | 2 |
| Cadherin signaling pathway | 4 |
| Circadian clock system | 1 |
| Cortocotropin releasing factor receptor signaling pathway | 3 |
| Cytoskeletal regulation by Rho GTPase | 3 |
| D1/D5 dopamine receptor mediated signaling pathway | 4 |
| D2/D3/D4 dopamine receptor mediated signaling pathway | 4 |
| EGF receptor signaling pathway | 2 |
| Endothelin signaling pathway | 4 |
| Enkephalin release | 3 |
| FAS signaling pathway | 1 |
| FGF signaling pathway | 3 |
| Folate biosynthesis | 1 |
| General transcription by RNA polymerase I | 1 |
| Glycolysis | 2 |
| Hedgehog signaling pathway | 1 |
| Heterotrimeric G-protein signaling pathway-Gi alpha and Gs | 11 |
| alpha mediated pathway | |
| Heterotrimeric G-protein signaling pathway-Gq alpha and Go | 9 |
| alpha mediated pathway | |
| Heterotrimeric G-protein signaling pathway-rod outer segment | 4 |
| phototransduction | |
| Huntington disease | 4 |
| Hypoxia response via HIF activation | 3 |
| Inflammation mediated by chemokine and cytokine signaling | 15 |
| pathway | |
| Insulin/IGF pathway-mitogen activated protein kinase kinase/ | 1 |
| MAP kinase cascade | |
| Insulin/IGF pathway-protein kinase B signaling cascade | 3 |
| Integrin signalling pathway | 7 |
| Interferon-gamma signaling pathway | 1 |
| Interleukin signaling pathway | 6 |
| Ionotropic glutamate receptor pathway | 2 |
| JAK/STAT signaling pathway | 1 |
| Metabotropic glutamate receptor group II pathway | 1 |
| Metabotropic glutamate receptor group III pathway | 2 |
| Muscarinic acetylcholine receptor 1 and 3 signaling pathway | 1 |
| Muscarinic acetylcholine receptor 2 and 4 signaling pathway | 1 |
| Nicotinic acetylcholine receptor signaling pathway | 2 |
| Notch signaling pathway | 2 |
| O-antigen biosynthesis | 2 |
| Ornithine degradation | 1 |
| Oxidative stress response | 3 |
| p53 pathway | 7 |
| p53 pathway feedback loops 2 | 2 |
| Parkinson disease | 7 |
| PDGF signaling pathway | 7 |
| Pentose phosphate pathway | 2 |
| Phenylethylamine degradation | 1 |
| PI3 kinase pathway | 2 |
| Ras Pathway | 4 |
| T cell activation | 6 |
| TGF-beta signaling pathway | 3 |
| Toll receptor signaling pathway | 1 |
| Transcription regulation by bZIP transcription factor | 2 |
| VEGF signaling pathway | 3 |
| Wnt signaling pathway | 9 |
| 5HT1 type receptor mediated signaling pathway | 4 |
| 5HT2 type receptor mediated signaling pathway | 5 |
| 5HT3 type receptor mediated signaling pathway | 1 |
| 5HT4 type receptor mediated signaling pathway | 3 |
| 5-Hydroxytryptamine degradation | 1 |
| Unclassified | 608 |

PANTHER pathway analysis mapped 70 genes with altered expression after TSA treatment to known signaling pathways important in cell proliferation and carcinogenesis. The number of affected genes involved in each pathway is listed above.

Predominant in these were pathways involved in carcinogenesis such as angiogenesis, apoptosis and more specifically the Ras, p53 and Wnt signaling cascades. While many of the genes have not been previously associated with medulloblastoma, pathways known to be involved in medulloblastoma pathogenesis such as sonic hedgehog signaling as well as EGF and IGF receptor tyrosine kinase signaling were also identified by the PANTHER analysis. In addition many TSA induced genes function in cerebellar development or possibly in medulloblastoma pathogenesis (Table 3).

TABLE 2
TSA induced genes in D283 cells with potential roles in
medulloblastoma pathogenesis.

| Gene | Biological Function |
|---|---|
| Notch1 | Neural differentiation |
| WIF1 | Wnt signaling/Developmental processes |
| DKK1 | Wnt signaling/Developmental processes |
| sFRP1 | Wnt signaling/Developmental processes |
| OLIG2 | mRNA transcription regulation |
| MYBL1 | Inhibition of apoptosis/Cell cycle control |
| CCNB3 | Cell cycle control/Cell proliferation and |
| | differentiation |
| LNK | Receptor protein tyrosine kinase/Calcium signaling |
| FBOXO33 | Transcription Factor |
| NDRG4 | Cell proliferation and differentiation |
| MAK | Protein phosphorylation |
| PAX6 | mRNA transcription regulation/Neurogenesis |
| NCAM1 | Cell adhesion-mediated signaling |
| ABCC2 | Small molecule transport/Detoxification |
| TMEFF2 | Oncogenesis |
| TPMT | Drug detoxification |
| CTGF | Receptor protein tyrosine kinase signaling pathway |
| INPP5F | Phospholipid metabolism |
| NRXN3 | Cell adhesion-mediated signaling |
| CYR61 | Angiogenesis/Cell cycle control |

For example PAX family gene expression has previously been associated with medulloblastoma. Similarly, Notch mediated signaling was recently associated with tumor formation in medulloblastoma mouse models

DKK1 is Down-Regulated in Medulloblastoma and Induced by HDAC Inhibition

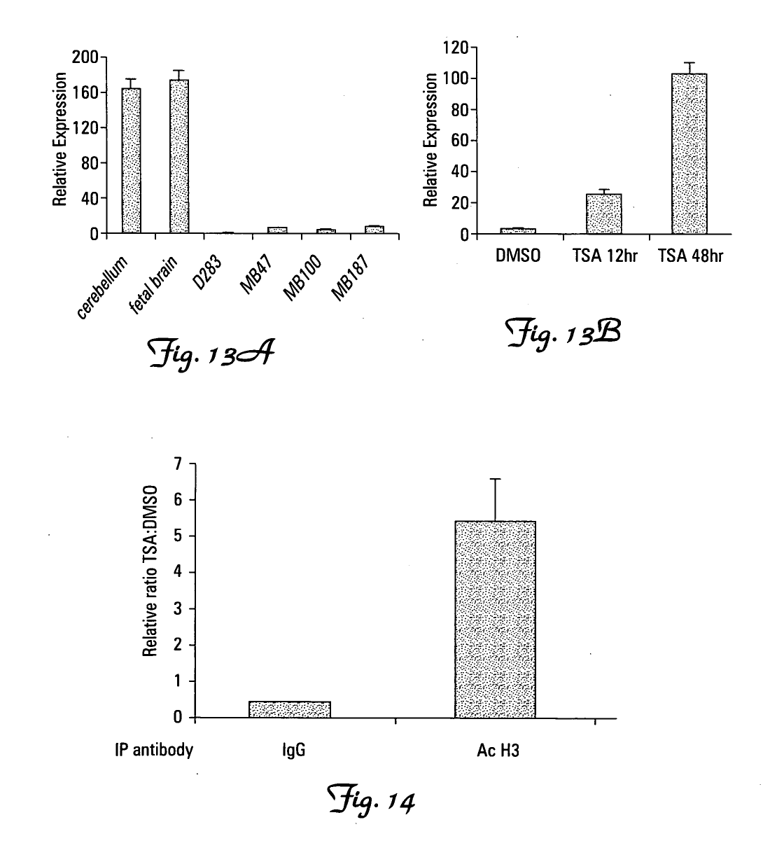

The goal of the inventors was to identify genes epigenetically silenced by histone deacetylation that are reversibly induced by TSA and thus are candidate tumor suppressor genes. Of 714 genes up-regulated on TSA treatment, the inventors found several genes that previously shown to suppress tumor growth in other cancers. Among these genes was DKK1, a Wnt antagonist that affects cell growth. The inventors examined changes in DKK1 expression on TSA treatment in three patient derived primary medulloblastoma cell lines (MB47, MB100 and MB187) and one immortalized cell line (D283) with respect to normal cerebellum by RT-PCR. DKK1 expression was significantly down-regulated in all cases, and increased on TSA treatment (FIGS. 13A and 13B).

To extend these findings to medulloblastoma tumors, the inventors compared DKK1 expression in ten patient tissue samples relative to normal cerebellum by RT-PCR. Box plot representation of ANOVA analysis showed significant decrease in DKK1 expression (p<0.001) in medulloblastoma patients (n=10) compared to normal cerebellum (n=3). When compared to normal cerebellum, all ten samples expressed 80% less DKK1. Analysis of variance confirmed that this difference was statistically significant (p<0.001).

Histone Acetylation Regulates DKK1 Expression in Medulloblastoma.

To further validate the role of histone tail modifications as an epigenetic silencing mechanism for DKK1 in medulloblastoma, the inventors performed chromatin immunoprecipitation (CHIP) using antibodies against acetylated histones H3 at Lys 9 position. Consistent with the inventors' earlier results, TSA treatment increased 5-fold the histone acetylation in the promoter region of DKK1 (FIG. 14). These data suggest that reversal of histone deacetylation by TSA was sufficient to allow DKK1 gene expression in medulloblastoma cells.

DKK1 Suppresses Medulloblastoma Growth and Induces Apoptosis.

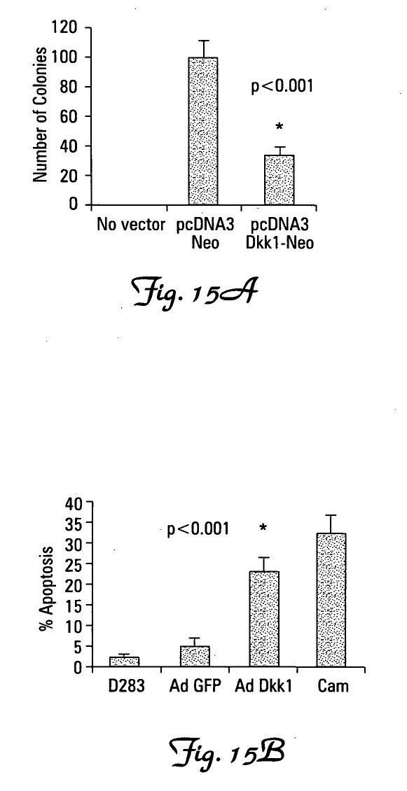

To test whether DKK1 can function as a tumor suppressor in medulloblastoma cells, its effect on growth was measured in colony focus-forming assays. Expression vectors were constructed that expressed the neomycin resistance gene along with DKK1. Vectors were transfected into D283 cells, selected in neomycin, and plated onto soft agar. DKK1 expression was confirmed by qPCR measurement of mRNA in control and DKK1 transfected cells After 3 weeks, cells expressing DKK1 formed 60% fewer neo-resistant colonies than controls (p<0.001) (FIG. 15A).

We next tested if DKK1 expression suppressed tumor development by growth inhibition or induction of tumor cell death. D283 cells were transduced with vectors expressing DKK1 and cell-cycle progression assayed. Efficiency of Ad-DKK1 infection was evaluated by GFP fluorescence and expression verified by qPCR. Ectopically expressing DKK1 did not affect cell cycle kinetics, suggesting that DKK1 inhibited growth did not occur via a block in cell cell-cycle progression (data not shown). In contrast, DKK1 enhanced apoptosis 4-fold in medulloblastoma cells as measured by annexin staining (FIG. 15B). These data support the hypothesis that DKK1 acts as a tumor suppressor gene in medulloblastoma.

Discussion

Epigenetic silencing of tumor suppressor genes control various aspects of carcinogenesis including proliferation, differentiation and apoptosis. This widespread mechanism has been implicated in regulating critical signaling cascades including Notch, sonic hedgehog and Wnt. Aberrant silencing of tumor suppressor genes has been associated with methylation of their promoter regions in medulloblastoma. Little is known however, about how epigenetic histone modifications may alter gene expression in medulloblastoma. Using D283 cells, a well characterized medulloblastoma cell line, we examined global epigenetic changes in medulloblastoma and identified genes belonging to multiple pathways important in tumorigenesis. Similar approaches in tumor cell lines by us and others have yielded several promising candidate tumor suppressor genes. In this screen, we identified DKK1, a Wnt signaling antagonist and confirmed it's silencing in medulloblastoma cell lines, primary tumor cells and medulloblastoma patient tissue.

The Wnt signaling pathway regulates multiple processes in development, tissue homeostasis and stem cell maintenance. Genetic mutations that disrupt Wnt signaling can cause tumors, the best studied case being colon adenocarcinoma. Although mutations in Wnt signaling components, APC, GSK3β and β-catenin have all been linked to colon cancer progression, mutations in these molecules occur only in a small subset of medulloblastoma patients, with most being the APC mutations in Turcot's syndrome. Our work demonstrates that Wnt signaling is also disrupted in medulloblastoma pathogenesis via the epigenetic silencing of DKK1.

We demonstrated that restoring DKK1 expression in medulloblastoma cells induced apoptosis and suppressed colony formation. Consistent with our data, others showed that expressing DKK1 in HeLa cells also suppressed transformation; and like our results, DKK1 inhibited growth by inducing apoptosis, not cell cycle arrest. In gliomas as well as models of ischemic neuronal apoptosis, DKK1 was also shown to be a pro-apoptotic factor. Thus DKK1's tumor suppressing activity is likely important in regulating proliferation in many cell types.

Our data raises two important questions with regard to DKK1 activity in medulloblastoma. The first is how DKK1 induces apoptosis in medulloblastoma. One possibility is that DKK1 suppresses the canonical Wnt signaling pathway thus down-regulating pro-survival molecules such as Bcl-2. Alternatively, DKK1 might stimulate pro-apoptotic pathways via non-canonical signaling mechanisms. Clues to DKK1 function in medulloblastoma might be provided by its role during vertebrate limb development where DKK1 inhibits pro-proliferative activities of canonical Wnt signaling and independently regulates apoptosis. Although the molecular mechanisms that allow DKK1 to regulate apoptosis are not well understood, some data suggests it regulates the JNK pathway. In mesothelioma, DKK1 antagonizes Wnt signaling in the absence of β-catenin by inducing JNK mediated apoptosis.

A second question is whether DKK1 is required for medulloblastoma tumor initiation or if it is associated with tumor progression. Recent evidence from colon cancer supports its role in tumor progression. Investigating DKK1 gene knockdown in mouse models of medulloblastoma will provide insight into its biological role in medulloblastoma tumorigenesis.

In this study we demonstrated the feasibility and robustness of a systematic approach to determine the role of epigenetically silenced genes in medulloblastoma. Our preliminary data suggest that DKK1 gene is a potent tumor suppressor and Wnt signaling is important in medulloblastoma pathogenesis, a factor not previously appreciated. We are now investigating the mechanistic basis of DKK1 activity in medulloblastoma. Recent studies indicate that Wnt signalling is negatively regulated by secreted Wnt antagonists such as secreted frizzled related proteins (sFRPs) and Dickkopfs (Dkks). We found Wif1 and SFRP1 also to be silenced in medulloblastoma cell lines and up-regulated on HDAC inhibition by TSA (data not shown). A systematic approach aimed to elucidate molecular mechanisms that various Wnt antagonists use to induce apoptosis in medulloblastoma may indicate new, more effective therapeutic targets. Similarly, studies with other epigenetically silenced genes will delineate their roles in malignant transformation and identify pathways involved in tumorigenesis.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A diagnostic method for predicting the recurrence of a brain tumor or cancer in a subject comprising comparing a test level value of a marker contained in a first physiological sample with a test level value of the marker from a second physiological sample, wherein the test level value of the marker from the second physiological sample of less than the test level value of the first physiological sample is predictive of the recurrence of a brain tumor or cancer in the subject, and wherein the marker is expressed from a nucleic acid encoding PCDH10, ITGA7, TNFRSF19, CDKN2D (p19), CDKN2B (p15), MAP2K3, BMP4, OLIG2, PAX6, HES1, TIMP3, TPMT, sFRP1, WIF1 or Dkk1.

2. The method of claim 1, wherein the tumor is a solid tumor.

3. The method of claim 2, wherein the solid tumor is a childhood tumor.

4. The method of claim 1, wherein the brain tumor is medulloblastoma.

5. The method of claim 1, wherein the first and second physiological samples are tissue samples.

6. The method of claim 1, wherein the first and second physiological samples are fluids.

7. The method of claim 6, wherein the fluid is blood or cerebrospinal fluid.

8. The method of claim 1, wherein the subject is a mammal.

9. The method of claim 1, wherein the subject is a human.

10. The method of claim 9, wherein the human is less than about 18 years old.

11. The method of claim 1, wherein the marker is a protein.

12. The method claim 1, wherein the marker is an RNA molecule.

13. The method of claim 1, wherein the marker is expressed from a nucleic acid encoding sFRP1.

14. The method of claim 1, wherein the marker is expressed from a nucleic acid encoding WIF1.

15. The method of claim 1, wherein the marker is expressed from a nucleic acid encoding Dkk1.

16. The method of claim 13, further comprising comparing a first level value of a marker expressed from a nucleic acid encoding WIF1 contained in the first physiological sample with a second level value of the marker expressed from a nucleic acid encoding WIF1 from the second physiological sample, wherein both (1) the test level value of the marker expressed from the nucleic acid encoding sFRP1 from the second physiological sample of less than the test level value of the marker expressed from the nucleic acid encoding sFRP1 from the first physiological sample and (2) the test level value of the marker expressed from the nucleic acid encoding WIF1 from the second physiological sample of less than the test level value of the marker expressed from the nucleic acid encoding WIF1 from the first physiological sample is predictive of the recurrence of a brain tumor or cancer in the subject.

17. The method of claim 13, further comprising comparing a first level value of a marker expressed from a nucleic acid encoding Dkk1 contained in the first physiological sample with a second level value of the marker expressed from a nucleic acid encoding Dkk1 from the second physiological sample, wherein both (1) the test level value of the marker expressed from the nucleic acid encoding sFRP1 from the second physiological sample of less than the test level value of the marker expressed from the nucleic acid encoding sFRP1 from the first physiological sample and (2) the test level value of the marker expressed from the nucleic acid encoding Dkk1 from the second physiological sample of less than the test level value of the marker expressed from the nucleic acid encoding Dkk1 from the first physiological sample is predictive of the recurrence of a brain tumor or cancer in the subject.

18. The method of claim 15, further comprising comparing a first level value of a marker expressed from a nucleic acid encoding WIF1 contained in the first physiological sample with a second level value of the marker expressed from a nucleic acid encoding WIF1 from the second physiological sample, wherein both (1) the test level value of the marker expressed from the nucleic acid encoding Dkk1 from the second physiological sample of less than the test level value of the marker expressed from the nucleic acid encoding Dkk1 from the first physiological sample and (2) the test level value of the marker expressed from the nucleic acid encoding WIF1 from the second physiological sample of less than the test level value of the marker expressed from the nucleic acid encoding WIF1 from the first physiological sample is predictive of the recurrence of a brain tumor or cancer in the subject.

19. The method of claim 13, further comprising comparing a first level value of a marker expressed from a nucleic acid encoding Dkk1 contained in the physiological sample with a second level value the marker expressed from a nucleic acid encoding Dkk1 from a physiological sample, wherein both (1) the test level value of the marker expressed from the nucleic acid encoding sFRP1 from the second physiological sample of less than the test level value of the marker expressed from the nucleic acid encoding sFRP1 from the first physiological sample, (2) the test level value of the marker expressed from the nucleic acid encoding WIF1 from the second physiological sample of less than the test level value of the marker expressed from the nucleic acid encoding WIF1 from the first physiological sample, and (3) the test level value of the marker expressed from the nucleic acid encoding Dkk1 of less than the first level value of the marker expressed from a nucleic acid encoding Dkk1 from the first physiological sample is predictive of the recurrence of a brain tumor or cancer in the subject.

20. A diagnostic method for determining the presence or recurrence of a brain tumor or cancer comprising comparing a test level value of a marker contained in a physiological sample from a subject suspected of having a brain tumor or cancer with a control level value of the marker, wherein a test level value of less than the control level value is predictive of the presence or recurrence of a brain tumor or cancer in the subject, and wherein the marker is expressed from a nucleic acid encoding PCDH10, ITGA7, TNFRSF19, CDKN2D (p19), CDKN2B (p15), MAP2K3, BMP4, OLIG2, PAX6, HES1, TIMP3, TPMT, sFRP1, WIF1 or Dkk1.

21. A method for treating a subject having a brain tumor or cancer, comprising administering to the subject an effective amount of a nucleic acid encoding PCDH10, ITGA7, TNFRSF19, CDKN2D (p19), CDKN2B (p15), MAP2K3, BMP4, OLIG2, PAX6, HES1, TIMP3, TPMT, sFRP1, WIF1 or Dkk1.

22. The method of claim 21, wherein the nucleic acid encodes sFRP1, WIF1 or Dkk1.

23. The method of claim 22, wherein the nucleic acid is contained in an expression cassette and is operably linked to a promoter.

24. The method of claim 23, wherein the expression cassette is contained in a vector.

25. The method of claim 24, wherein the vector is a viral vector.

26. A kit for determining the presence or recurrence of a brain tumor or cancer in a subject containing packaging material and a means for detecting at least one marker that is expressed from a nucleic acid encoding sFRP1, WIF1 or Dkk1 and instructions for use as a kit for diagnosing or predicting the recurrence of a brain tumor or cancer in the subject.
